(19) United States
(12) Patent Application Publication
(10) Pub. No.: US 2023/0201284 A1
(43) Pub. Date: Jun. 29, 2023

(54) Uses and Formulations of Cannabinoids

(71) Applicant: ADD Advanced Drug Delivery Technologies Ltd., Pratteln (CH)

(72) Inventors: Reinhard Nowak, Binzen (DE); Mirko Nowak, Lörrach (DE); Jesko Jay Nowak, Lörrach (DE); Norbert Pöllinger, Müllheim (DE)

(73) Assignee: ADD Advanced Drug Delivery Technologies Ltd., Pratteln (CH)

(21) Appl. No.: 17/923,749

(22) PCT Filed: May 11, 2020
(86) PCT No.: PCT/EP2020/063087

§ 371 Date: Nov. 7, 2022

(51) Int. Cl. A61K 36/185; A61K 47/38

(52) U.S. Cl. CPC A61K 36/185; A61K 47/38

(57) ABSTRACT

Uses and formulations of cannabinoids, in particular of cannabidiol, are provided.

The cannabinoids, in particular cannabidiol, are used for the treatment of patients suffering from inflammatory conditions associated with autoimmune diseases, chronic inflammatory diseases and inflammatory conditions in connection with infections, including cytokine release syndrome (CRS).

Formulations are especially for oral administration of cannabinoids, in particular of cannabidiol. These formulations are useful for treating patients suffering from conditions as referred to above.

The present application claims priority from PCT Patent Application No. PCT/EP2020/063087 filed on May 11, 2020, the disclosure of which is incorporated herein by reference in its entirety.

### FIELD OF THE INVENTION

The present invention relates to uses and formulations of cannabinoids, in particular of cannabidiol. According to the invention, the cannabinoids, in particular cannabidiol, are used for the treatment of patients suffering from inflammatory conditions characterised by elevated IL-6 levels. This includes inflammatory conditions associated with autoimmune diseases, chronic inflammatory diseases and inflammatory conditions in connection with infections, including cytokine release syndrome (CRS).

The invention also provides formulations for oral administration of cannabinoids, in particular of cannabidiol. These formulations are useful for treating patients suffering from inflammatory conditions.

### BACKGROUND OF THE INVENTION

Inflammatory conditions associated with autoimmune diseases, chronic inflammatory diseases and inflammatory conditions in connection with infections, including cytokine release syndrome (CRS), present a significant disease burden for the afflicted patients. Some conditions can even be life threatening.

While various treatments for such conditions have been suggested, there still remains a need for further treatment options, in particular for a simple and convenient pharmacological intervention.

Independent of the above considerations cannabinoids and in particular cannabidiol have been considered as drugs. There is evidence that cannabinoids can be beneficial for treating a number of clinical conditions, including pain, inflammation, epilepsy, sleep disorders, indication of multiple sclerosis, anorexia, and schizophrenia (N. Bruni et al., Cannabinoid Delivery Systems for Pain and Inflammation Treatment. Molecules 2018, 23, 2478).

While the use of cannabinoids in various indications has been suggested, but so far only limited applications received market authorisation.

### SUMMARY OF THE INVENTION

An objective of the invention is to provide compositions and treatment regimens for the treatment of patients suffering from inflammatory conditions characterised by elevated IL-6 levels.

According to the invention such compositions and treatment regimens are provided.

The cannabinoid is preferably administered orally. It is administered at a dose between 250 mg and 5000 mg one to four times per day.

The cannabinoid can be formulated as a solid dispersion, in particular a solid dispersion comprising the cannabinoid and a solubilizer which is an amphiphilic block copolymer capable of forming a micellar solution if combined with an aqueous medium.

The block copolymer is preferably a poloxamer.

The cannabinoid can also be incorporated in a formulation comprising a core and a coating on the core, wherein the coating comprises the cannabinoid, one or more water-soluble film formers and not more than 20 wt-%, based on the weight of all components, other excipients.

Further objectives and their solution can be concluded from the detailed description of the invention below.

### BRIEF DESCRIPTION OF THE FIGURES

With reference to the figures the invention is explained in more detail below.

FIG. 1 schematically shows the preparation of a solid dispersion containing a cannabinoid and the interaction of the solid dispersion with aqueous media.

FIG. 2 shows the in vitro release from three pellet products comprising 2-[1R-3-methyl-6R-(1-methylethenyl)-2-cyclohexen-1-yl]-5-pentyl-1,3-benzenediol as active substance and low-viscosity hydroxypropylmethyl cellulose as film former.

### DETAILED DESCRIPTION OF THE INVENTION

Interleukins (ILs) are a group of cytokines, i.e., secreted proteins which act as signal molecules. The function of the immune system depends in a large part on interleukins.

One of the interleukins is Interleukin-6 (IL-6). By activating different kinase pathways IL-6 promotes complex biologic reactions such as cell proliferation, cell differentiation, oxidative stress and immune regulation.

IL-6, which acts as a pro-inflammatory cytokine, has important roles in both innate and adaptive immunity.

IL-6 can be produced by different cell types, among them macrophages, endothelial cells, and T cells. The production of IL-6 can be initiated in reaction to infection. IL-6 is also formed in response to certain other cytokines, such as tumour necrosis factor (TNF).

IL-6 plays a role in the innate immune system, contributing to the acute phase response. IL-6 acts on hepatocytes to induce expression of C-reactive protein (CRP), fibrinogen, and serum amyloid A.

IL-6 also plays a key role in the adaptive immune response, mediating proliferation of antibody-producing B cells. In consequence, an enhanced antibody response is observed. IL-6 furthermore acts synergistically with IL-1B and TNF-α stimulating T cell activation, growth and differentiation.

In non-infectious inflammations, such as inflammations caused by burn or traumatic injury, damage-associated molecular patterns (DAMPS) originating from damaged or dying cells stimulate Toll-like receptors, which leads to the production of IL-6.

While IL-6 has important physiological roles. dysregulation of this cytokine is implicated in the onset and development of several disease states. Dysregulated IL-6 production has been demonstrated to play a pathological role in various autoimmune and inflammatory diseases. Targeting IL-6 is a rational approach to the treatment of these diseases.

Patients to be Treated

Patients to be treated according to the present invention suffer from inflammatory conditions associated with autoimmune diseases, chronic inflammatory diseases and inflammatory conditions in connection with infections, including cytokine release syndrome (CRS).

IL-6 plays a crucial role in inflammatory conditions associated with autoimmune diseases. More in particular, IL-6 together with TGF-ß promotes differentiation of IL-17-producing T helper cells (Th17) that play a crucial role in the induction of autoimmune tissue injury. At the same time, IL-6 inhibits TGF-ß-induced regulatory T cell (Treg) differentiation. Thus, IL-6-induces dominance of Th17 cells over Treg cells.

The resultant Th17/Treg imbalance leads to breakage of immunological tolerance and is of pathological importance for the development of various autoimmune and inflammatory diseases.

IL-6 is elevated in numerous chronic inflammatory disorders.

Clinical trials of tocilizumab, a humanized anti-IL-6 receptor antibody have verified its efficacy and tolerable safety for patients with rheumatoid arthritis, and systemic juvenile idiopathic arthritis.

In an activated memory T cell line, CBD dose-dependently reduces the autoantigen-specific Th17 cell phenotype as shown by a decrease of the Th17 signature cytokine IL-17. The reduction is accompanied by decreased IL-6 production and secretion and increased production of IL-10, critical changes associated with reduced Th17 cell propagation (E. Kozela et al. (2013). Cannabinoids decrease the th17 inflammatory autoimmune phenotype. J Neuroimmune Pharmacol 8(5): 1265-76).

Further, cannabinoids, in particular CBD, suppress circulating IL-6 in various animal models of diseases involving an inflammatory phenotype including diabetes, asthma, pancreatitis and hepatitis (see J. M. Nichols and B. L. F. Kaplan (2020). Immune responses regulated by cannabidiol. Cannabis and Cannabinoid Research 5(1): 12-31).

Thus, according to the present invention, inflammatory conditions characterised by elevated IL-6 levels can be treated by administration of cannabinoids, in particular cannabidiol.

These conditions can also involve autoimmune components.

Conditions with or without demonstrated autoimmune component which can be treated according to the invention are rheumatic diseases. Rheumatic diseases include osteoarthritis; rheumatoid arthritis; fibromyalgia; systemic lupus erythematosus; gout; juvenile idiopathic arthritis; infectious arthritis; psoriatic arthritis; polymyositis; bursitis; ankylosing spondylitis; reactive arthritis; scleroderma; polymyalgia rheumatica.

A further condition that can be treated is giant cell arteritis (GCA).

Still further, inflammatory bowel disease (IBD) can be treated according to the invention.

IL-6 is also produced by adipocytes. In patients suffering from metabolic syndrome, serum IL-6 levels are increased. This results in chronic inflammatory processes, which in turn lead to atherosclerosis, insulin tolerance and coagulation disorders. According to the present invention, patients suffering from metabolic syndrome are treated. The treatment prevents, halts or ameliorates the results of the chronic inflammatory processes. The treatment in particular prevents, halts or ameliorates atherosclerosis, insulin tolerance and/or coagulation disorders.

In infectious diseases, early after infection, the immune response is essential to eliminate the infectious agent and to prevent progression to more severe disease stages. Strategies to boost immune responses at this stage may be important. Immunosuppressive therapies are expected to endanger the patient in this early disease phase.

If the early immune response is impaired or insufficient, the infectious agent will propagate and then cause massive tissue damage, eventually leading to inflammation caused by pro-inflammatory cytokines. The damaged cells as a consequence result in innate inflammation largely mediated by pro-inflammatory macrophages and granulocytes. IL-6 levels are elevated in patients with infection.

IL-6 levels are in particular elevated in with septicemia and sepsis. IL-6 levels are correlated with severity of sepsis, as assessed by clinical and laboratory parameters.

CRS can occur in a number of infectious and non-infectious diseases. CRS is a form of systemic inflammatory response syndrome. Immune cells are activated by stressed or infected cells through receptor-ligand interactions. CRS occurs when large numbers of white blood cells are activated to release inflammatory cytokines, which in turn activate more white blood cells in a positive feedback loop of pathogenic inflammation, leading to a rapid elevation of pro-inflammatory cytokines.

The term cytokine storm is used for severe cases of CRS.

Patients have classical serum biomarkers of CRS including elevated CRP, LDH, IL-6, and ferritin.

Patients requiring intensive care typically have higher blood concentrations of pro-inflammatory cytokines than those not requiring intensive care. Patients will in particular show increased of the pro-inflammatory cytokine IL-6 levels. Increased levels soon after onset of the disease indicate a severe course of disease. CRS itself is considered to be the cause of several pathological events.

A high level of IL-6 is a hallmark and important driving force of the CRS.

The present invention is based on the finding that pharmacological intervention can prevent or reduce unwanted components of the immune response.

This is achieved by a pharmacological intervention counteracting the release of pro-inflammatory cytokines, in particular IL-6.

The invention in particular allows preventing or ameliorating the cytokine release syndrome (CRS) and its clinical manifestations, including unwanted inflammatory processes.

The present invention provides a simple and convenient treatment for the above discussed conditions, namely a treatment which can be administered orally.

Suitable criteria for initiating treatment are based on laboratory findings.

Laboratory findings upon which treatment of a patient may be initiated include one or more of serum IL-6≥5.4 pg/ml; CRP level>70 mg/L (without other confirmed infectious or non-infectious course); CRP level>−40 mg/L and doubled within 48 hours (without other confirmed infectious or non-infectious course); lactate dehydrogenase>250 U/L; D-dimer>1 μg/mL; serum ferritin>300 μg/mL.

Preferably, treatment initiation is based on an increased level of IL-6.

Further, treatment of a patient may be initiated if the patient, optionally in addition to one of the above criteria, shows thrombocytopenia<120.000×10E9/L, and/or a lymphocyte count<0.6×10E9/L.

Treatment progress can be monitored by reduction of IL-6, CRP, transaminases, LDH, D-dimer, ferritin, IL-18, IL-18, interferon gamma, neutrophils, lymphocytes, neutrophil-to-lymphocyte ratio (NLR) in %, for instance between first dose, day 14 and day 28.

The treatment is continued until relevant clinical improvements are achieved. In conditions involving chronic inflammation, treatment may be long-term.

Active Ingredients

Cannabinoids are a heterogeneous group of pharmacologically active substances that have an affinity for the so-called cannabinoid receptors. The cannabinoids include, for example, tetrahydrocannabinol (THC) and the non-psychoactive cannabidiol (CBD).

Cannabinoids can be both phytocannabinoids and synthetic cannabinoids.

Phytocannabinoids are a group of about 70 terpenophenolic compounds (V. R. Preedy (ed.), Handbook of Cannabis and Related Pathologies (1997)). These compounds typically contain a monoterpene residue that is attached to a phenolic ring and has a C₃-C₅ alkyl chain that is in the meta position to the phenolic hydroxyl group.

A preferred group of cannabinoids are tetrahydrocannabinols with the following general formula (1):

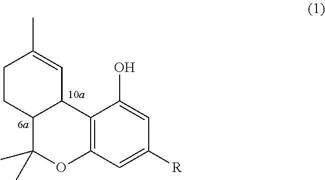

wherein R is selected from among C₁-C₂₀-alkyl, C₂-C₂₀-alkenyl or C₂-C₂₀-alkynyl, and optionally has one or more substituents.

In a further preferred group of compounds of the above general formula (1), R is selected from among C₁-C₁₀-alkyl or C₂-C₁₀-alkenyl, and optionally has one or more substituents.

In particular, in formula (1) R is an alkyl radical with the formula C₅H₁₁.

Compounds of general formula (1) can be present in the form of stereoisomers. The centres 6a and 10a preferably each have the R configuration.

The tetrahydrocannabinol is in particular A9-THC with the chemical name (6aR,10aR)-6,6,9-trimethyl-3-pentyl-6a, 7,8,10a-tetrahydro-6H-benzo[c]chromene-1-ol. The structure is reflected by the following formula (2):

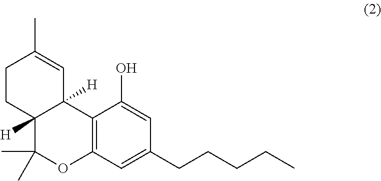 [SMILES: CCCCCc1cc(O)c2c(c1)OC(C)(C)[C@@H]1CCC(C)=C[C@@H]21]

Another preferred group of cannabinoids are cannabidiols with the following general formula (3):

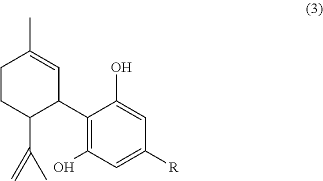

wherein R is selected from among C₁-C₂₀-alkyl, C₂-C₂₀-alkenyl or C₂-C₂₀-alkynyl, and optionally has one or more substituents.

In a further preferred group of compounds having the general formula (3) above, R is selected from among C₁-C₁₀-alkyl or C₂-C₁₀-alkenyl, and optionally has one or more substituents.

In particular, R in formula (3) is an alkyl radical with the formula C₅H₁₁.

The cannabidiol is in particular 2-[(1R,6R)-3-methyl-6-(1-methylethenyl)-2-cyclohexen-1-yl]-5-pentyl-1,3-benzenediol. In the present specification, if the term cannabidiol or its abbreviation CBD is used, this particular compound is meant, unless stated otherwise.

CBD is a major constituent of Cannabis sp.—besides the psychotropic A9-THC. The psychotropic effect of THC is mediated by the cannabinoid receptor CB1 that is mainly expressed on neurons. In contrast to THC, CBD is a peripherally and centrally acting compound without psychotropic activity.

According to the invention, a combination of A9-THC ((6aR, 10aR)-6,6,9-trimethyl-3-pentyl-6a,7,8,10a-tetrahydro-6H-benzo[c]chromen-1-ol) and CBD (2-[(1R,6R)-3-methyl-6-(1-methylethenyl)-2-cyclohexen-1-yl]-5-pentyl-1,3-benzenediol) can be used.

Another preferred group of cannabinoids are cannabinols with the following general formula (4):

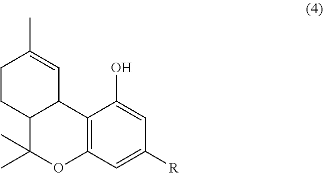

wherein R is selected from among C₁-C₂₀-alkyl, C₂-C₂₀-alkenyl or C₂-C₂₀-alkynyl, and optionally has one or more substituents.

In a further preferred group of compounds having the general formula (4) above, R is selected from among C₁-C₁₀-alkyl or C₂-C₁₀-alkenyl, and optionally has one or more substituents.

In particular, in formula (4) R is an alkyl radical having the formula C₅H₁₁.

The cannabinol is especially 6,6,9-trimethyl-3-pentyl-6H-dibenzo[b,a]pyran-1-ol.

According to the invention, cannabinoids or cannabinoid mixtures of hemp extracts can also be used.

For example, Nabiximols is a plant extract mixture used as a drug of the leaves and flowers of the hemp plant (Cannabis sativa L.) with standardized contents of tetrahydrocannabinol (THC) and cannabidiol (CBD).

Synthetic cannabinoids can also be used.

These include 3-(1,1-dimethylheptyl)-6,6a,7,8,10,10a-hexahydro-1-hydroxy-6,6-dimethyl-9H-dibenzo[b,d]pyran-9-one. This compound contains two stereogenic centres. The drug nabilone is a 1:1 mixture (racemate) of the (6aR,10aR) form and the (6aS,10aS) form. Nabilone is a preferred cannabinoid according to the invention.

Another example of a synthetic cannabinoid is JWH-018 (1-naphthyl-(1-pentylindol-3-yl)methanone).

The use of cannabinoids, in particular of cannabidiol, is based on their pharmacodynamic properties. Cannabinoid receptors include CB1, which is predominantly expressed in the brain, and CB2, which is primarily found on the cells of the immune system. The fact that both CB1 and CB2 receptors have been found on immune cells suggests that cannabinoids play an important role in the regulation of the immune system. Independent of this finding, several studies show that cannabinoids downregulate cytokine and chemokine production and, in some models, upregulate T-regulatory cells (Tregs) as a mechanism to suppress inflammatory responses. The endocannabinoid system is also involved in immunoregulation.

Cannabinoids, in particular cannabidiol, are in particular suitable for preventing or at least halting or significantly slowing down progression of inflammatory conditions associated with autoimmune diseases, chronic inflammatory diseases and inflammatory conditions in connection with infections, including cytokine release syndrome (CRS).

This therapeutic utility is based on the pharmacodynamic properties of the cannabinoids, especially their interaction with the endocannabinoid system and further pharmacological targets including serotonergic receptors, adenosine signalling, vanilloid receptors, PPAR-γ receptors and GPR55, which has been shown to be immune-modulating or even immune-suppressive.

Cannabinoids, in particular cannabidiol, exert effects on the innate immune system (i.e., the part of the immune system enabling a fast reaction to pathogens via neutrophils, macrophages and other myeloid cells). Affected cell types of the innate immune system in particular include mononuclear cells, macrophages, neutrophils, dendritic cells, microglial cells and myeloid-derived suppressor cells (MDSCs) J. M. Nichols and B. L. F. Kaplan (2020), loc. it.):
The release of pro-inflammatory cytokines in human mononuclear cells is suppressed by nanomolar or micromolar concentrations of CBD.
CBD (20 mg/kg) decreases the number of leukocytes including macrophages and neutrophils in the bronchoalveolar lavage fluid of mice after LPS-induced lung inflammation. This effect is mediated by the adenosine A2A receptor (A. Ribeiro et al. (2012). Cannabidiol, a non-psychotropic plant-derived cannabinoid, decreases inflammation in a murine model of acute lung injury: role for the adenosine A(2A) receptor. Eur J Pharmacol 678(1-3): 78-85). Furthermore, CBD also inhibits the migration of human neutrophils (D. McHugh et al. (2008). Inhibition of human neutrophil chemotaxis by endogenous cannabinoids and phytocannabinoids: evidence for a site distinct from CB1 and CB2. Mol Pharmacol 73(2): 441-50). Reduction in neutrophil count is of therapeutic relevance.
CBD suppresses the CD83 dendritic cell activation marker on dendritic cells derived from individuals with human immune deficiency virus (HIV) infection, but not healthy individuals (A. T. Prechtel and A. Steinkasserer (2007). CD83: an update on functions and prospects of the maturation marker of dendritic cells. Arch Dermatol Res 299(2): 59-69).
CBD (1-16 μmol/l) induces apoptosis in microglial cells, the main innate immune cells of the central nervous system (H. Y. Wu et al. (2012). Cannabidiol-induced apoptosis in murine microglial cells through lipid raft. Glia 60(7): 1182-90).
The numbers of natural killer (NK) cells and natural killer T (NKT) cells are not affected by CBD (5 mg/kg per day) or even increased (2.5 mg/kg per day) in healthy rats, suggesting that CBD may enhance the NK/NKT-related non-specific immune response (B. Ignatowska-Jankowska et al. (2009). Cannabidiol-induced lymphopenia does not involve NKT and NK cells. J Physiol Pharmacol 60 Suppl 3: 99-103).
Additionally, CBD is able to induce the regulatory immune cell population of MDSCs. In mice with chemically induced acute hepatitis, CBD (25 mg/kg) induces the expression of MDSCs, along with a reduction of pro-inflammatory cytokines such as IL-2, TNF-α and IL-6; the effect is mediated by the TRPV1 receptor (V. L. Hegde et al. (2011). Role of myeloid-derived suppressor cells in amelioration of experimental autoimmune hepatitis following activation of TRPV1 receptors by cannabidiol. PLoS One 6(4): e18281).

In addition, cannabinoids, in particular CBD, exhibit an effect on cells of the adaptive immune system. The adaptive immune system is comprised of T and B cells. T cells either directly lyse or induce apoptosis of infected cells (cytotoxic T cells) or recruit other immune cells (T helper [Th] cells) including B cells that produce antibodies against pathogens:
In a study with healthy rats, daily administration of 5 mg/kg CBD significantly reduced the number of T cells including T helper cells and cytotoxic T cells and of B cells (B. Ignatowska-Jankowska et al., loc. it.).
It has been suggested that a shift from Th1 to Th2 immune response resulting in decreased pro-inflammatory cytokines such as TNF-α and IL-12 and increased anti-inflammatory cytokines such as IL-10 accounts for CBD's anti-inflammatory effects (L. Weiss et al. (2006). Cannabidiol lowers incidence of diabetes in non-obese diabetic mice. Autoimmunity 39(2): 143-51).
In an activated memory T cell line, CBD dose-dependently (1-5 μmol/l) reduced the autoantigen-specific Th17 cell phenotype as shown by a decrease of the Th17 signature cytokine IL-17. The finding was accompanied by decreased IL-6 production and secretion and increased production of IL-10, critical changes associated with reduced Th17 cell propagation (E. Kozela et al. (2013), loc. it.).
CBD was shown to induce regulatory T cells (Tregs) in several disease models J. M. Nichols and B. L. F. Kaplan (2020), loc. it.). In mice with ischemia-reperfusion-induced kidney injury, levels of regulatory T-17 (Treg17) cells were decreased and Th17 levels were increased. The physiological function of Treg17 cells includes the inhibition of Th17-mediated inflammatory actions. A dose of 10 mg/kg CBD after induced kidney injury was renoprotective and reversed these effects (B. Baban et al. (2018). Impact of cannabidiol treatment on regulatory T-17 cells and neutrophil polarization in acute kidney injury. Am J Physiol Renal Physiol 315(4): F1149-f58).

Many studies demonstrate that cannabinoids and in particular CBD exert their immune suppressive and anti-inflammatory effects by the suppression of pro-inflammatory cytokines such as TNF-α, IFN-γ, IL-6, IL-1p, IL-2, IL-17A, and of chemokines, such as CCL-2. The pro-inflammatory cytokine IL-6 has a central role in inflammatory conditions associated with autoimmune diseases, chronic inflammatory diseases and inflammatory conditions in connection with infections, including cytokine release syndrome (CRS). IL-6 signalling is among the main canonical pathways affected by cannabinoids and in particular CBD. Since cannabinoids and in particular CBD suppress circulating IL-6 in various inflammation animal models, suppression of IL-6 thereby preventing unwanted immune and inflammatory reactions is considered the most relevant mode of action of cannabinoids and in particular CBD in patients as considered herein.

According to the present invention, a cannabinoid, in particular cannabidiol, can also be applied as part of a combination treatment.

Dosing and Administration

According to the invention, the cannabinoid, in particular cannabidiol, is preferably administered orally.

Other routes of administration are, however, also contemplated, in particular for patients who cannot take an oral medication. Such other routes are in particular intravenous, intramuscular or subcutaneous injection.

The administration is in one to four doses per day. Typically, the administration is twice per day (BID).

According to the invention, patients are treated with an effective dose of the cannabinoid, in particular cannabidiol.

A single dose may be between 250 mg and 5000 mg, administered one to four times per day, for instance, BID.

Exemplary doses are 375 mg, 750 mg, 1500 mg, and 3000 mg, administered one to four times per day, for instance, BID.

A particularly preferred dose is 1500 mg, administered one to four times per day, preferably, BID.

As indicated above, cannabinoids, in particular cannabidiol, have suppressive pharmacodynamic effects on the immune system in various animal models.

It has been shown in divergent animal models that in the majority of cases inflammatory processes are suppressed by doses between 2.5 and 20 mg/kg body weight mostly administered intraperitoneally or orally. Alternative routes have been transdermal, intranasal and IV application J. M. Nichols and B. L. F. Kaplan BLF (2020), loc. it.).

In cellular models determining a suppressive effect on IL-6 secretion in the majority of cases the effective concentration was in a magnitude of 5 μM (J. Chen et al. (2016). Protective effect of cannabidiol on hydrogen peroxide induced apoptosis, inflammation and oxidative stress in nucleus pulposus cells. Mol Med Rep 14(3): 2321-7).

Based on the molecular weight of CBD of 314.5 g/mol the resulting concentration is 1,570 ng/ml.

Ribeiro et al. investigated the influence of CBD on LPS-induced acute lung injury in mice as disease model for ARDS, once in a prophylactic intervention (A. Ribeiro et al. (2012), loc. it.) and once in the acute phase as a therapeutic intervention (A. Ribeiro et al. (2014). Cannabidiol improves lung function and inflammation in mice submitted to LPS-induced acute lung injury. Immunopharmacol Immunotoxicol 37(1): 35-41).

Mice were prophylactically administered 0.3, 1.0, 10, 20, 30, 40 and 80 mg/kg CBD via the intraperitoneal route. 60 minutes after administration acute lung injury was induced via intranasal instillation of Escherichia coli LPS. Mice were killed 1, 2, 4 and 7 days after instillation. Total leukocytes migration, myeloperoxidase activity, pro-inflammatory cytokine production including TNF-α and IL-6 and vascular permeability were significantly decreased (A. Ribeiro et al. (2012), loc. it.). Effects were dose dependent but reached a nearly maximum extent with 20 mg/kg in this study with prophylactic application.

In a subsequent study the same group investigated the effect of CBD after acute lung injury had been induced by LPS. The testing scenario was similar except for the time point of intervention which was chosen as 6 h after LPS installation. Doses of 20 and 80 mg/kg were chosen based on the results of the earlier study (A. Ribeiro et al. (2014), loc. it.). The study showed an improved mechanical lung function, decreased leukocyte migration (neutrophil, macrophages and lymphocytes) into the lungs, decreased myeloperoxidase activity in the lung tissue, reduced vascular permeability and production of proinflammatory cytokines/chemokines at 20 mg/kg.

A comparative investigation for systemic exposure after i.p. and oral application of CBD in mice and rats has shown that 120 mg/kg as a single dose leads to a maximum plasma concentration of 14,000 ng/ml in mice (S. Deiana et al. (2012). Plasma and brain pharmacokinetic profile of cannabidiol (CBD), cannabidivarine (CBDV), Delta(9)-tetrahydrocannabivarin (THCV) and cannabigerol (CBG) in rats and mice following oral and intraperitoneal administration and CBD action on obsessive-compulsive behaviour. Psychopharmacology (Berl) 219(3): 859-73).

Taking these data into consideration and assuming a dose-proportional relationship for the resulting plasma concentrations, a dose of 20 mg/kg, shown to be effective in the animal model, leads to a target peak exposure of 2,300 ng/ml.

As regards the systemic exposure data in humans, after fasted administration of Epidyolex® morning maximum values under steady-state conditions of 541 ng/ml are observed. Evening maximum values are higher. A factor of 3.8 in systemic exposure is observed between morning and evening upon twice daily Epidyolex® administration (L. Taylor et al. (2018). A Phase I, Randomized, Double-Blind, Placebo-Controlled, Single Ascending Dose, Multiple Dose, and Food Effect Trial of the Safety, Tolerability and Pharmacokinetics of Highly Purified Cannabidiol in Healthy Subjects. CNS Drugs 32(11): 1053-67).

Thus, the standard dose of 1,500 mg CBD administered twice daily as already approved with Epidyolex® is considered safe and efficacious.

Based on the above data, patients will also benefit from other doses in the range outlined herein.

Galenics

Low and variable bioavailability of cannabinoids, in particular upon oral administration, hampers effective clinical use of these compounds.

Cannabinoids, in particular cannabidiol, are difficult to formulate due to their highly lipophilic nature.

In fact, cannabinoids are highly lipophilic molecules (log P 6-7) with very low water solubility (2-10 μg/ml). The log P is the decimal logarithm of the n-octanol/water partition coefficient. The partition coefficient can be determined experimentally. Values typically refer to room temperature (25° C.). The partition coefficient can also be roughly calculated from the molecular structure.

In addition to poor solubility, cannabinoids, in particular CBD, are subject to high first-pass metabolism, which further contributes to poor systemic availability after oral administration.

Various formulations of cannabinoids have been suggested.

Due to the high lipophilicity of cannabinoids, salt formation (i.e. pH adjustment), cosolvency (e.g. ethanol, propylene glycol, PEG400), micellization (e.g. Polysorbate 80, Cremophor-ELP), emulsification including micro and nano emulsification, complexation (e.g. cyclodextrins) and encapsulation in lipid-based formulations (e.g. liposomes) are among the formulation strategies considered in the prior art. Nanoparticle systems have also been proposed (N. Bruni et al., loc. it.).

Various solid oral dosage forms have been proposed in the patent literature, for example in WO 2008/024490 A2 and in WO 2018/035030 A1. These documents do not contain data on release behaviour, so the practical suitability of the proposed forms for the administration of cannabinoids remains unclear.

WO 2015/065179 A1 describes compressed tablets which, in addition to cannabidiol, contain lactose and sucrose fatty acid monoesters.

Dronabinol (A9-THC) is marketed in the form of capsules (Marinol) and as an oral solution (Syndroso). The Marinol® capsules are soft gelatine capsules containing the active ingredient in sesame oil.

The drug product Sativex® containing nabiximols is a mouth spray that is sprayed onto the inside of the cheek.

Self-emulsifying drug delivery systems (SEDDS) which are mixtures of oils, surfactants and optionally contain hydrophilic solvents have also gained interest in an approach to improve the oral bioavailability of certain cannabinoids (K. Knaub et al. (2019). A Novel Self-Emulsifying Drug Delivery System (SEDDS) Based on VESIsorb® Formulation Technology Improving the Oral Bioavailability of Cannabidiol in Healthy Subjects. Molecules, 24(16), 2967). Upon contact with an aqueous phase, such as gastric or intestinal fluids, SEDDS spontaneously emulsify under conditions of gentle agitation.

VESIsorb®, a self-emulsifying drug delivery formulation technology developed by Vesifact AG (Baar, Switzerland) has shown increased oral bioavailability of certain lipophilic molecules.

The preparation Epidiolex® recently approved by the US-FDA as an orphan drug for the treatment of certain forms of epilepsy is provided in the form of an oral solution that in addition to the active ingredient cannabidiol contains the excipients absolute ethanol, sesame oil, strawberry aroma and sucralose.

Notwithstanding all these proposals, however, there is still a need for improved dosage forms for cannabinoids, such as cannabidiol, in particular for solid oral dosage forms.

Various approaches suggested in the prior art are not entirely satisfactory. Some of these approaches rely on liquid formulations. Handling of such formulations is more difficult than that of solid dosage form. Prior art formulations are often complex to prepare and sometimes lead to only low bioavailability of the cannabinoid.

While formulations known in the art may be used in the treatment aspects of the present invention, the invention also provides improved formulations.

In one aspect of the present invention, a formulation is provided which is a solid dispersion comprising a cannabinoid, in particular cannabidiol, and a solubilizer. As further detailed below, solid dosage forms for oral administration showing satisfactory bioavailability can be obtained in this way.

According to this aspect, a highly lipophilic cannabinoid, like the almost water insoluble CBD, is combined with a solubilizer in order to increase the drug solubility by solubilization in aqueous media. An increased solubility will in turn increase the absorption rate of the drug compound.

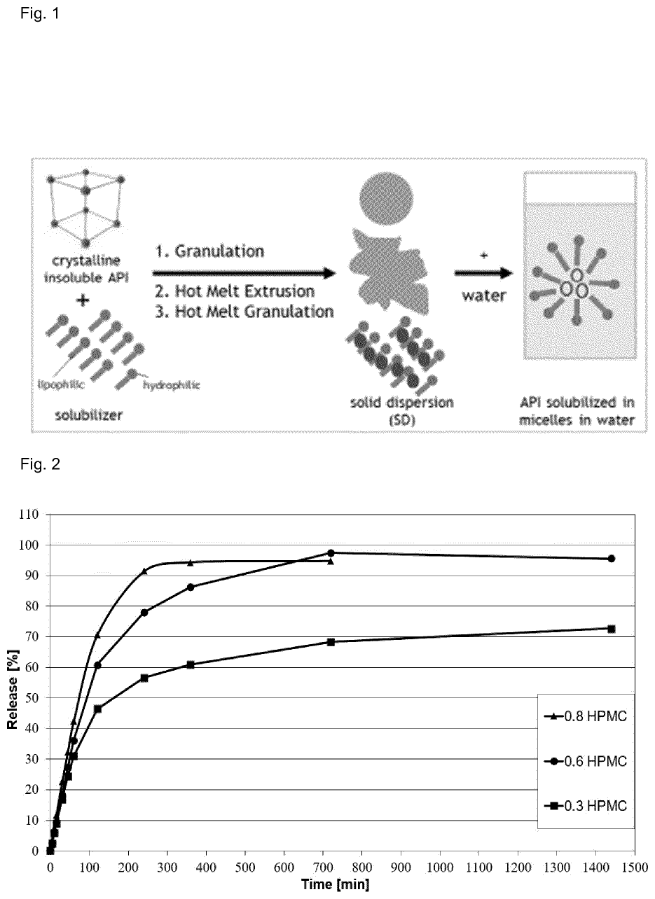

The solid dispersion comprising a cannabinoid, in particular cannabidiol, and a solubilizer leads to the formation of micelles upon contact with water or other aqueous media, such as gastrointestinal fluids. The micelles are essentially formed from the drug substance, surrounded by solubilizer (see FIG. 1).

One aspect of the invention is accordingly a micellar composition comprising an aqueous phase in which micelles are dispersed, which micelles comprise a cannabinoid, in particular cannabidiol, and a solubilizer.

Suitable solubilizers are solid at ambient temperature. They have surfactant properties and, if used in appropriate concentration ranges in aqueous media, in particular water, can form micellar solutions.

Suitable solubilizers include in particular amphiphilic block copolymers.

More in particular, block copolymers containing at least one polyoxyethylene block and at least one polyoxypropylene block can be used.

Suitable block copolymers are in particular poloxamers. Poloxamers are block copolymers whose molecular weights range from 1,100 to over 14,000. Different poloxamers differ only in the relative amounts of propylene and ethylene oxides added during manufacture.

Poloxamers have the following general formula:

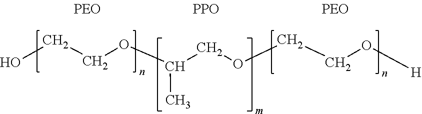

In this general formula, n designates the number of polyoxyethylene units, m designates the number of polyoxypropylene units.

In one embodiment, the solubilizer is Poloxamer 188 (Kolliphor P188; former brand name Lutrol F 68)/BASF; CAS No.: 9003-11-6).

Kolliphor P188 is a polyoxyethylene-polyoxypropylene block copolymer of the above general formula wherein n is approximately 79 and m is approximately 28.

Kolliphor P188 is available as a white to slightly yellowish waxy substance in the form of micropearls having a melting point of 52-57° C. It meets the requirements of Ph. Eur., USP/NF for Poloxamer 188.

The solid dispersion can be prepared by a hot melt process. The cannabinoid and the solubilizer are heated to a temperature which allows forming a homogenous melt in which the cannabidiol and the solubilizer are present in a molecular state before they form a solid dispersion when cooled.

The melt is processed into pellets. This can be carried out by batch-wise spray granulation/pelletisation (fluid bed topspray, Wurster=bottomspray technology).

Alternatively, and preferably, continuous spray granulation/pelletisation (fluid bed MicroPx Technology, ProCell Technology) is used.

An alternative preparation method relies on dispersing the cannabinoid, in particular cannabidiol, in an aqueous solution of the solubilizer, for instance, in a solution of the solubilizer in water.

The solution can be processed by batch-wise spray granulation/pelletisation (fluid bed topspray or Wurster=bottomspray technology) or preferably by continuous spray granulation/pelletisation (fluid bed MicroPx Technology, ProCell Technology) to obtain a solid granulate.

The formulation may contain one or more excipients in addition to the active ingredient and the solubilizer. It is in particular considered to include an antioxidant or a combination of antioxidants to protect the cannabinoid, in particular cannabidiol, from oxidation.

Useful antioxidants include ascorbylpalmitate, alpha-tocopherol, butylhydroxytoluol (BHT, E321), butylhydroxyanisol (BHA, E320), ascorbic acid, and ethylenediaminetetraacetic acid (EDTA) sodium.

The antioxidant or combination of antioxidants may be added to the melt or the solution of the solubiliser prior to the addition of cannabinoid, in particular CBD.

The solid dispersion preferably does not contain more than 20% by weight, relative to all components, of additional excipients.

The solid dispersion is preferably free or essentially free of triglycerides. Essentially free means that the formulation contains less than 5% by weight, relative to all components, of triglycerides.

Further, the solid dispersion is preferably free or essentially free of fatty acids. Essentially free means that the formulation contains less than 5% by weight, relative to all components, of fatty acids.

The solid dispersion granules or pellets can be filled into hard gelatine capsules, sachets or stick packs using commercial standard technology and equipment.

Depending on the final dosage strength per unit, the solid dispersion granules can be filled into capsules which are feasible for swallowing (e.g. capsule size 2-1 for 25 mg/dose). Alternatively, for high dosed units, bigger capsules can be used as a primary packaging material for the granules. Such capsules are not for swallowing (e.g. capsule size up to 000/sprinkle caps for 100-200 mg/dose). Rather, the solid dispersion granules are to be sprinkled on food or dispersed in a liquid, e.g., water.

A composition obtained by dispersing the solid dispersion granules in a liquid can be applied to patients being not able to swallow by means of a syringe through a gastric tube.

Alternatively, the solid dispersion granules can also be processed into tablets. The solid dispersion granules are combined with one or more excipients, such as a disintegrant, a glidant, and/or a lubricant. The obtained mixture is then compressed into tablets.

According to another aspect of the invention a product for the release of a cannabinoid, in particular cannabidiol, comprises a core and a coating on the core, wherein the coating comprises the cannabinoid, in particular cannabidiol, one or more highly lipophilic physiologically active substances, one or more water-soluble film formers and no more than 20 wt-% of other excipients, based on the weight of all components.

Surprisingly, it was found that solid oral dosage forms of cannabinoids, in particular cannabidiol, can be provided, wherein the release can be controlled with the help of the amount of film-forming agent (s) relative to the amount of the cannabinoid.

The use of one or more film formers not only allows for the formation of a coating containing the cannabinoid, but also serves to control the release. In particular, a film former promotes the release of the cannabinoids which are only sparingly soluble in water. Only by means of the film former, these are released in sufficient quantity and speed.

For this purpose, a core is provided with a coating which, in addition to a cannabinoid, in particular cannabidiol, comprises one or more water-soluble film formers. In addition to the cannabinoid(s), the coating preferably does not contain any other physiologically active substances.

Examples of suitable water-soluble film formers are methyl cellulose (MC), hydroxypropyl methyl cellulose (HPMC), hydroxypropyl cellulose (HPC), hydroxyethyl cellulose (HEC), sodium carboxymethyl cellulose (Na-CMC) and polyvinyl pyrrolidone (PVP).

Hydroxypropylmethyl cellulose (HPMC), in particular low-viscosity HPMC, such as HPMC with a viscosity of a 2% (w/w) aqueous solution at 20° C. of 6 mPa s or less is preferred.

An HPMC with a viscosity of a 2% (w/w) aqueous solution at 20° C. of 3 mPa s, as is available under the trade name Pharmacoat® 603, is especially preferred.

The coating of a cannabinoid and one or more water-soluble film formers may contain other commonly used excipients. According to the invention, the quantity of further excipients is limited to not more than 20 wt-%, based on the weight of all components. Preferably, no more than 10 wt-%, based on the weight of all components, of further excipients is comprised.

In a particularly preferred embodiment, the coating consists of cannabinoid(s) and film former(s).

Pellets according to the invention have a coating which contains one or more water-soluble film formers, based on the total amount of cannabinoid, in a total amount of 0.1-10 wt-%, preferably in a total amount of 0.5-8 wt-%, and in particular in a total proportion of 1-6 wt.-%.

It is assumed that if the amount of film former is too small, the release takes place only very slowly and incompletely. By selecting the proportion in the specified ranges the release of the physiologically active substance can be adjusted. For example, the release from an oral dosage form can be adjusted so that the physiologically active substance is released over the conventional time of the gastrointestinal passage.

The coating is applied to cores. The cores may have any structure and may consist of any physiologically acceptable materials. For example, tablets, mini-tablets, pellets, granules or crystals may be used as cores. The cores may contain or consist of, for example, sugar, tartaric acid or microcrystalline cellulose. Inert starter cores, such as pellets made of microcrystalline cellulose, are preferred. Such pellets are commercially available under the name Cellets®.

The size of the cores is not limited. Suitable sizes are in the range from 10 μm to 2000 μm, for example in the range from 50 μm to 1500 μm and preferably 100 μm to 1000 μm, the size may be determined by sieve analysis. In particular, pellets from a sieve fraction of 500-710 μm may be used.

The products according to the invention can be produced by first producing a spray liquid which contains one or more cannabinoids and one or more water-soluble film formers.

Since cannabinoids have only a very low solubility in water, an organic solvent or a mixture of an organic solvent and water is typically used.

The spray liquid is then applied to cores. The liquid components are evaporated, so that a coating is formed on the cores that is mostly free of solvents and water. This may be done, for example, in a fluidized bed system, a jet bed system, a spray dryer or a coater.

Coated cores may then be used as an oral dosage form. Coated pellets may e.g. be offered in sachets, or they may be processed further.

The cores coated according to the invention may also be provided with one or more further coatings. This enables additional control of the release.

In a preferred embodiment, no further coating controlling the release is provided.

Coated pellets may also be used to obtain multiparticulate dosage forms. For example, they can be filled into capsules or incorporated into tablets. In one embodiment, they are processed into orally dispersible tablets.

Coated pellets with different release profiles may be combined in one dosage form (capsule/tablet/sachet). The products according to the invention release the cannabinoid contained therein or, if more than one cannabinoid is contained, all cannabinoids contained therein after ingestion in the digestive tract. The products are especially used for controlled release. They, in particular, release more than 30 wt-% and less than 80 wt-% of the physiologically active substance contained within two hours. In addition, they, especially, release more than 40 wt-% and less than 90 wt-% of the physiologically active substance contained within three hours. Furthermore, they release more than 50 wt-% and less than 95 wt-% of the physiologically active substance contained within four hours. If more than one cannabinoid is comprised, the information relates to all substances contained.

In each case the release is determined in a blade stirrer apparatus in 1000 ml of phosphate buffer pH 6.8 with an addition of 0.4% Tween® 80 at 37° C.

### EXAMPLES

The invention is illustrated with the help of specific examples, without being restricted in any way thereby.

### Example 1

A cannabidiol containing granulate (solid dispersion) can be obtained using 20 parts by weight of cannabidiol and 80 parts by weight of Kolliphor P188. For preparing the granulate, the following options are available.

Option (a)

The components are heated to a temperature of about 100° C. The melt is sprayed onto a solid sample of CBD in a fluidised bed at a product temperature of about 15-25° C. For this batch process, topspray, bottomspray and tangential spray configurations can be used.

Option (b)

The components are heated to a temperature of about 100° C. The melt is sprayed into a fluidised bed apparatus which is initially empty. Solidification of the melt under fluidised bed conditions with a product temperature of about 15-25° C. leads to the formation of a granulate. For this batch process, topspray, bottomspray and tangential spray configurations can be used.

Option (c)

Preparation of a granulate from a melt can also be carried out continuously. This can be done by using the ProCell or MicroPx Technology (Glatt).

Option (d)

The melt can also be processed in a spray tower. Using prilling nozzles, spherical particles of defined size can be obtained.

### Example 2

A cannabidiol containing granulate (solid dispersion) can be obtained using 30 parts by weight of cannabidiol and 70 parts by weight of Kolliphor P188. For preparing the granulate, the options outlined in Example 1 are available.

### Example 3

A cannabidiol containing granulate (solid dispersion) can be obtained using 40 parts by weight of cannabidiol and 60 parts by weight of Kolliphor P188. For preparing the granulate, the options outlined in Example 1 are available.

### Example 4

A cannabidiol containing granulate (solid dispersion) can be obtained using 20.05 parts by weight of cannabidiol, 76 parts by weight of Kolliphor P188, 3.4 parts by weight of Avicel PH 101, 0.5 parts by weight of Aerosil 200 and 0.05 parts by weight of BHT.

A melt from Kolliphor P188 and BHT having a temperature of about 100° C. is sprayed onto a solid CBD, Avicel PH 101 and Aerosil 200 in a fluidised bed. The product temperature is about 15-25° C. For this batch process, topspray, bottomspray and tangential spray configurations can be used.

### Example 5

Compositions based on different weight ratios of CBD/solubilizer were prepared by melting and cooling the melts. The compositions were analyzed in terms of in vitro dissolution in 0.1N HCl following the USP paddle method.

For comparison the oily Cannabidiol solution according to DAC/NRF 22.10. and the commercial product Bionic Softgels was also tested.

CBD release after 60 min of in vitro dissolution testing in 0.1N HCl:

CBD/Kolliphor P188=33/67; 200 mg CBD: 69% drug release

CBD/Kolliphor P188=27/73; 200 mg CBD: 82% drug release

CBD/Kolliphor P188=20/80; 200 mg CBD: 96% drug release

CBD in oily (Miglyol 812) solution; 200 mg CBD: 0% drug release

Bionic Softgels; 25 mg CBD 96% drug release

### Example 6

Tablets are prepared using 93.5 wt % of a granulate according to one of Examples 1 to 4, 5 wt % Polyplasone XL (disintegrant), 1% Aerosil 200 (glidant) and 0.5% magnesium stearate (lubricant).

### Example 7

Pellets were made using the quantities of ingredients shown in Table 1 below.

For this purpose, 2-[1R-3-methyl-6R-(1-methylethenyl)-2-cyclohexen-1-yl]-5-pentyl-1,3-benzenediol (Canapure PH) was dissolved in ethanol 96%. This active ingredient has a log P of about 6.1.

Another solution was prepared by dissolving HPMC (Pharmacoat® 603) in water.

The HPMC solution was then gradually added to the cannabidiol solution.

Then amorphous silicon dioxide (Syloid® 244 FP) was added.

It was stirred with a propeller stirrer.

The spray liquid obtained was sprayed onto starter cores made of microcrystalline cellulose (Cellets® 500).

This was done in a Mini-Glatt fluidized bed system with a Wurster insert. The inlet air temperature was 40° C. The average spray rate was 0.5 g/min.

TABLE 1
Substances and quantities used

| | Formulation | | | | |
|---|---|---|---|---|---|
| | HPMC 0.8 | HPMC 0.6 | | HPMC 0.3 | |
| | Quantity | Quantity | | Quantity | |
| Solids | | | | | |
| Cellets 500 | 60.01 g/81.5% | 60.00 g/72.7% | | 60.00 g/72.7% | |
| Canapure PH | 21.02 g/16.1% | 21.00 g/24.2% | | 21.26 g/24.5% | |
| Pharmacoat 603 | 1.05 g/0.8% | 0.53 g/0.6% | | 0.26 g/0.3% | |
| Syloid 244 FP | 2.10 g/1.6% | 2.10 g/2.4% | | 2.10 g/2.4% | |
| Liquids (not | | | | | |
| included in | | | | | |
| the product) | | | | | |
| Ethanol 96% | 79.81 g | 79.83 | g | 79.82 | g |
| Pure water | 25.20 g | 25.21 | g | 25.21 | g |
| Spray liquid | | | | | |
| Solid content | 18.71% | 18.36% | | 18.36% | |
| (wt./wt.) | | | | | |
| Quantity sprayed | 72.80 g | 122.50 | g | 122.50 | g |

TABLE 2
Products

| Formulation | HPMC 0.8 | HPMC 0.6 | HPMC 0.3 |
|---|---|---|---|
| Theoretical | 73.63 g | 82.49 g | 82.49 g |
| yield | | | |
| Practical yield | 64.30 g/87.33% | 75.03 g/90.95% | 74.24 g/90.00% |
| Coating weight | 31.49% | 66.82% | 63.31% |
| gain | | | |

### Example 8

The release from the pellet products obtained in Example 1 is examined using a blade stirrer apparatus in 1000 ml phosphate buffer pH 6.8 with an addition of 0.4% Tween® 80, specifically at 37° C. The results obtained are shown in FIG. 2.

1. A cannabinoid for treatment of a patient suffering from an inflammatory condition characterised by elevated IL-6 levels.

2. The cannabinoid according to claim 1, wherein the cannabinoid is cannabidiol (2-[(1R,6R)-3-methyl-6-(1-methylethenyl)-2-cyclohexen-1-yl]-5-pentyl-1,3-benzenediol).

3. The cannabinoid according to claim 1, wherein the patient suffers from an inflammatory condition associated with an autoimmune disease.

4. The cannabinoid according to claim 1, wherein the patient suffers from a chronic inflammatory disease.

5. The cannabinoid according to claim 1, wherein the patient suffers from an inflammatory condition in connection with an infection.

6. The cannabinoid according to claim 5, wherein the treatment is for preventing or ameliorating cytokine release syndrome (CRS).

7. The cannabinoid according to claim 1, wherein the condition to be treated is a rheumatic disease.

8. The cannabinoid according to claim 7, wherein the disease is selected from osteoarthritis; rheumatoid arthritis; fibromyalgia; systemic lupus erythematosus; gout; juvenile idiopathic arthritis; infectious arthritis; psoriatic arthritis; polymyositis; bursitis; ankylosing spondylitis; reactive arthritis; scleroderma; polymyalgia rheumatica.

9. The cannabinoid according to claim 1, wherein the condition to be treated is giant cell arteritis (GCA).

10. The cannabinoid according to claim 1, wherein the condition to be treated is inflammatory bowel disease (IBD).

11. The cannabinoid according to claim 1, wherein the patient suffers from metabolic syndrome.

12. The cannabinoid according to claim 11, wherein treatment prevents, halts or ameliorates atherosclerosis, insulin tolerance and/or coagulation disorders.

13. The cannabinoid according to claim 1, wherein the treatment reduces the serum IL-6 level.

14. The cannabinoid according to claim 1, wherein the treatment is initiated based on one or more of serum IL-6≥5.4 pg/ml; CRP level>70 mg/L (without other confirmed infectious or non-infectious course); CRP level>=40 mg/L and doubled within 48 hours (without other confirmed infectious or non-infectious course); lactate dehydrogenase>250 U/L; D-dimer>1 μg/mL; serum ferritin>300 μg/mL.

15. The cannabinoid according to claim 1, wherein the treatment is initiated if the patient shows thrombocytopenia<120.000×10E9/L, and/or a lymphocyte count<0.6×10E9/L.

16. The cannabinoid according to claim 1, wherein the treatment is initiated if the patient shows at least one laboratory finding selected from serum IL-6≥5.4 pg/ml; CRP level>70 mg/L (without other confirmed infectious or non-infectious course); CRP level>=40 mg/L and doubled within 48 hours (without other confirmed infectious or non-infectious course); lactate dehydrogenase>250 U/L; D-dimer>1 μg/mL; serum ferritin>300 μg/mL; and shows thrombocytopenia<120.000×10E9/L, and/or a lymphocyte count<0.6×10E9/L.

17. The cannabinoid according to claim 1, wherein the treatment is initiated if the serum IL-6≥5.4 pg/ml.

18. The cannabinoid according to claim 1, wherein the cannabinoid is administered orally.

19. The cannabinoid according to claim 1, wherein the cannabinoid is administered at a dose between 250 mg and 5000 mg one to four times per day.

20. The cannabinoid according to claim 19, wherein the dose is 375 mg, 750 mg, 1500 mg, or 3000 mg, and this dose is administered one to four times per day.

21. The cannabinoid according to claim 20, wherein the dose is administered BID.

22. The cannabinoid according to claim 1, wherein the cannabinoid is administered BID at a dose of 1500 mg.

23. The cannabinoid according to claim 1, wherein the cannabinoid is formulated as a solid dispersion.

24. The cannabinoid according to claim 23, wherein the solid dispersion comprises the cannabinoid and a solubilizer which is an amphiphilic block copolymer capable of forming a micellar solution if combined with an aqueous medium.

25. The cannabinoid according to claim 23, wherein the solubilizer is a block copolymer containing at least one polyoxyethylene block and at least one polyoxypropylene block.

26. The cannabinoid according to claim 25, wherein the solubilizer is a poloxamer.

27. The cannabinoid according to claim 26, wherein the formulation comprises cannabidiol as the active substance, polaxamer 188 as the solubilizer and optionally an antioxidant.

28. The cannabinoid according to claim 23, wherein the formulation, when subjected to an in vitro dissolution test in 0.1N HCl following the USP paddle method, releases at least 60 wt % of the cannabinoid within 60 minutes.

29. The cannabinoid according to claim 1, wherein the cannabinoid is incorporated in a formulation comprising a core and a coating on the core, wherein the coating comprises the cannabinoid, one or more water-soluble film formers and not more than 20 wt-%, based on the weight of all components, other excipients.

30. The cannabinoid according to claim 29, wherein hydroxypropylmethyl cellulose (HPMC) is used as the water-soluble film former.

31. The cannabinoid according to claim 29, wherein the film former/film formers, based on the total amount of cannabinoid, is/are comprised in a total proportion of 0.3-10 wt-%.

32. The cannabinoid according to claim 29, wherein more than 30 wt-% and less than 80 wt-% of the cannabinoid contained is released within two hours; and/or wherein more than 40 wt-% and less than 90 wt-% of the cannabinoid contained is released within three hours; and/or wherein more than 50 wt-% and less than 95 wt-% of the cannabinoid contained is released within four hours.
